(19) United States
(12) Patent Application Publication
(10) Pub. No.: US 2012/0205274 A1
(43) Pub. Date: Aug. 16, 2012

(54) ALLOGRAFT BONE COMPOSITION HAVING A GELATIN BINDER

(75) Inventors: Moon Hae Sunwoo, Old Tappan, NJ (US); Arthur A. Gertzman, Flemington, NJ (US); Barbara L. Merboth, Bridgewater, NJ (US)

(73) Assignee: Musculoskeletal Transplant Foundation

(21) Appl. No.: 13/456,461

(22) Filed: Apr. 26, 2012

Related U.S. Application Data
(63) Continuation-in-part of application No. 10/150,097, filed on May 20, 2002, now Pat. No. 7,045,141.
Application No. 10/150,097 is a continuation-in-part of application No. 12/478,044, filed on Jun. 4, 2009, now abandoned.
Application No. 12/478,044 is a continuation of application No. 11/433,362, filed on May 15, 2006, now abandoned.
Application No. 11/433,362 is a continuation-in-part of application No. 10/150,097, filed on May 20, 2002, now Pat. No. 7,045,141.

(51) Int. Cl. A61K 35/32; A61P 19/00; A61J 1/00; A61K 9/00

(52) U.S. Cl. 206/438; 424/549; 424/400

(57) ABSTRACT

The invention is directed toward an osteoimplant for application to a bone defect site to promote new bone growth at the site which comprises a new bone growth inducing composition of demineralized allograft bone material mixed with an aqueous phosphate buffered gelatin which when lyophilized to remove water from the composition crosslinks the gelatin to form a solid structure and when rehydrated is flexible

### RELATED APPLICATIONS

This is a continuation-in-part of U.S. patent application Ser. No. 10/150,097 filed May 20, 2002 which will issue into U.S. Pat. No. 7,045,141 on May 16, 2006.

### STATEMENT REGARDING FEDERALLY SPONSORED RESEARCH OR DEVELOPMENT

Not applicable.

### REFERENCE TO SEQUENCE LISTING, A TABLE OR A COMPUTER PROGRAM LISTING COMPACT DISC APPENDIX

None.

### FIELD OF INVENTION

The present invention is generally directed toward a surgical bone defect filling product and more specifically to a shaped bone implant using allograft bone and gelatin with the gelatin being cross linked by lyophilization of the composition to form a solid composition which is later rehydrated for application to a bone defect area.

### BACKGROUND OF THE INVENTION

Surgical implants should be designed to be biocompatible in order to successfully perform their intended function. Biocompatibility may be defined as the characteristic of an implant acting in such a way as to allow its therapeutic function to be manifested without secondary adverse affects such as toxicity, foreign body reaction or cellular disruption.

Many products have been developed in an attempt to develop bone deficit fillers. One such example is autologous bone particles or segments recovered from the patient. When removed from the patient, the segments or bone particles are wet and viscous from the associated blood. This works very well to heal the defect but requires significant secondary surgery resulting in lengthening the surgery, extending the time the patient is under anesthesia and increasing the cost. In addition, a significant increase in patient morbidity is attendant in this technique as the surgeon must take bone from a non-involved site in the patient to recover sufficient healthy bone, marrow and blood to perform the defect filling surgery. This leads to significant post-operative pain.

Another product group involves the use of inorganic materials to provide a matrix for new bone to grow at the surgical site. These inorganic materials include hydroxyapatite obtained from sea coral or derived synthetically. Either form may be mixed with the patient's blood and/or bone marrow to form a gel or a putty. Calcium sulfate or plaster of Paris may be mixed with water to similarly form a putty. These inorganic materials are osteoconductive but are bioinert. The calcium sulfate materials absorb slowly but the other materials do not absorb or become remodeled into natural bone. They consequently remain in place indefinitely as a brittle, foreign body in the patient's tissue.

Allograft bone is a logical substitute for autologous bone. It is readily available and precludes the surgical complications and patient morbidity associated with autologous bone as noted above. Allograft bone is essentially a collagen fiber reinforced hydroxyapatite matrix containing active bone morphogenic proteins (BMP) and can be provided in a sterile form. The demineralized and partially demineralized form of allograft bone is naturally both osteoinductive and osteoconductive. The demineralized allograft bone tissue is fully incorporated in the patient's tissue by a well established biological mechanism. It has been used for many years in bone surgery to fill the osseous defects previously discussed.

Demineralized allograft bone is usually available in a lyophilized or freeze dried in sterile form to provide for extended shelf life. The bone in this form is usually very coarse and dry and is difficult to manipulate by the surgeon. One solution to use such freeze dried bone has been provided in the form of a gel, GRAFTON®, a registered trademark of Osteotech Inc., which is a simple mixture of glycerol and lyophilized, demineralized bone powder having little to no residual calcium, averaging less than 0.01% and having a particle size in the range of 0.1 cm to 1.2 cm (1000 microns to 12,000 microns) as is disclosed in U.S. Pat. No. 5,073,373.

GRAFTON works well to allow the surgeon to place the allograft bone material at the site. However, the carrier, glycerol has a very low molecular weight (92 Daltons) and is very soluble in water, the primary component of the blood which flows at the surgical site. Glycerol also experiences a marked reduction in viscosity when its temperature rises from room temperature (typically 22° C. in an operating room) to the temperature of the patient's tissue, typically 37° C. This combination of high water solubility and reduced viscosity causes the allograft bone material with a glycerol carrier to be “runny” and to flow away from the site almost immediately after placement; this prevents the proper retention of the bone material within the site as carefully placed by the surgeon. Furthermore concerns about the neurotoxic behavior of glycerol have been noted in Spine Vol. 26, No. 13 Jul. 1, 2001 in an editorial by the Deputy Editor, C. A. Dickman, M.D. which has a clinical recommendation to limit the dose of GRAFTON®, avoid use in certain medical situations, avoid use with small children and to avoid direct contact of GRAFTON® with exposed spinal nerves.

These problems with GRAFTON gel have been attempted to be resolved by using a much larger particle size of allograft bone, specifically lamellae or slivers of bone created by milling or slicing the bone before mixing it with the glycerol carrier. This improves both the bulk viscosity and the handling characteristics of the mixture but still leaves the problem of the fast rate of dissipation of the carrier and some bone due to the solubility of the glycerol carrier.

U.S. Pat. No. 5,290,558 discloses a flowable demineralized bone powder composition using an osteogenic bone powder with large particle size ranging from about 0.1 to about 1.2 cm. mixed with a low molecular weight polyhydroxy compound possessing from 2 to about 18 carbons including a number of classes of different compounds such as monosaccharides, disaccharides, water dispersible oligosaccharides and polysaccharides.

Hence, the advantages of using the smaller bone particle sizes as disclosed in the 5,073,373 gel patent were compromised by using bone lamellae in the shape of threads or filaments and retaining the low molecular weight glycerol carrier. This later prior art is disclosed in U.S. Pat. Nos. 5,314,476 and 5,507,813 and the tissue forms described in these patents are known commercially as the GRAFTON® Putty and Flex, respectively.

The use of the very low molecular weight glycerol carrier also requires a very high concentration of glycerol to be used to achieve the bulk viscosity. Glycerol and other similar low molecular weight organic solvents are toxic and irritating to the surrounding tissues.

U.S. Pat. No. 5,356,629 discloses making a rigid gel in the nature of a bone cement to fill defects in bone by mixing biocompatible particles, preferably polymethylmethacrylate coated with polyhydroxyethylmethacrylate in a matrix selected from a group which lists hyaluronic acid to obtain a molded semi-solid mass which can be suitably worked for implantation into bone. The hyaluronic acid can also be utilized in monomeric form or in polymeric form preferably having a molecular weight not greater than about one million Daltons. It is noted that the nonbioabsorbable material which can be used to form the biocompatible particles can be derived from xenograft bone, autogenous bone as well as other materials. The bioactive substance can also be an osteoinductive agent such as demineralized bone powder, in addition to morselized cancellous bone, aspirated bone marrow and other autogenous bone sources. The average size of the particles employed is preferably about 0.1 to about 3.0 mm, more preferably about 0.2 to about 1.5 mm, and most preferably about 0.3 to about 1.0 mm. It is inferentially mentioned but not taught that particles having average sizes of about 7,000 to 8,000 microns, or even as small as about 100 to 700 microns can be used. However, the biocompatible particles used in this reference are used in a much greater weight ranging from 35% to 70% by weight then that taught by the present invention. The reference is directed toward a cement used for implantation of hip prosthesis and is not used to promote bone growth.

U.S. Pat. No. 5,830,493 is directed toward a composite porous body (hyaluronic acid listed in a group of compounds) comprising a porous frame and a surface layer comprising a bioabsorbable polymer material formed on the surface. A bone morphogenetic protein (BMP) is carried on the surface and inside of the composite porous body. There is no use of demineralization of bone.

U.S. Pat. No. 5,053,049 discloses a composition for treating bone defects comprising demineralized bone osteogenic powder that has been tanned and used with any suitable biologically compatible or inert carrier which may include polysaccharides. The tanning can be by glutaraldehyde or different agents including formaldehyde or alcohol.

Another attempt to solve the bone composition problem is shown in U.S. Pat. No. 4,172,128 which discloses demineralized bone material mixed with a carrier to reconstruct tooth or bone material by adding a mucopolysaccharide to a mineralized bone colloidal material. The composition is formed from a demineralized coarsely ground bone material, which may be derived from human bones and teeth, dissolved in a solvent forming a colloidal solution to which is added a physiologically inert polyhydroxy compound such as mucopolysaccharide or polyuronic acid in an amount which causes orientation when hydrogen ions or polyvalent metal ions are added to form a gel. The gel will be flowable at elevated temperatures above 35 C and will solidify when brought down to body temperature. Example 25 of the patent notes that mucopolysaccharides produce pronounced ionotropic effects and that hyaluronic acid is particularly responsible for spatial cross-linking. Unfortunately this bone gel is difficult to manufacture and requires a premolded gel form.

U.S. Pat. No. 4,191,747 teaches a bone defect treatment with coarsely ground, denatured bone meal freed from fat and ground into powder. The bone is not demineralized and retains its complete mineral content. The bone meal is mixed with a polysaccharide in a solution of saline and applied to the bone defect site.

U.S. Pat. No. 5,854,207 is directed to a composition containing a morphogenic protein stimulatory factor which is vacuum dried to create a cross link.

U.S. Pat. No. 5,707,962 discloses a bone repair composition having matrix of organic or inorganic materials such as ceramic or synthetic polymer. The preferred embodiment uses collagen and demineralized bone particles.

U.S. Pat. No. 5,510,418 discloses binding glycosaminoglycan to hydrophilic synthetic polymers such a polyethylene glycol by specific chemical bonds to provide bone repair compositions.

U.S. Pat. No. 4,440,750 discloses the use of demineralized osteogenic bone powder in a physiological carrier such as saline to treat a bone defect site to promote new bone growth.

Another prior art product is the formulation of demineralized allograft bone particles in collagen. Both bovine and human collagen have been used for this application. Bovine collagen carries the risk of an immunogenic reaction by the recipient patient. Recently, it has been found that a disease of cattle, bovine spongioform encephalopathy (mad cow disease) is transmitted from bovine tissue to humans. Thus, bovine tissue carries a risk of disease transmission and is not a desirable carrier for allograft tissue.

Human collagen is free of these animal based diseases. However, collagen absorbs slowly in the human body, particularly in a bony site with usually a low degree of vascularity. The slow absorption of collagen can delay the growth of new bone and result in the formation of scar tissue at the site. This could result in a non-bony healing and a result with much less tensile strength.

All of the previous noted products are in a paste or gel form and when set into a body cavity are shortly washed or carried away from the site by body fluids. An attempt to overcome this problem is set forth in U.S. Pat. No. 6,294,187 which discloses a compressed load bearing composition of bone particles with a bulk density of greater than about 0.7 g/cm3 and a wet compressive strength of at least about 3 MpA

Accordingly, the prior art as embodied in the glycerol and other carrier based technology to deliver demineralized and mineralized allograft bone to a surgical osseous site is replete with problems and only partially addresses the problems inherent in the correcting surgical defects which are solved in the present invention.

### SUMMARY OF THE INVENTION

The subject shaped implant is a complex formulation of a partially demineralized bone matrix (DBM) mixed with a gelatin and saline phosphate buffer acting as a carrier for the agent, DBM which is placed in a mold resulting in a desired implant shape such as a strip, wedge or the like. The shaped implant is then lyophilized for 24 to 33 hours to remove from 90% to 99%+ of the water from the composition. The composition is cross linked by lyophilization to form a solid strip which can be made flexible by controlled hydration to produce a flexible, strong suturable strip which is used as a spinal fusion device particularly for posteralaterial spinal fusion. The strip or other shaped implant presents the DBM, and its bone morphogenetic proteins (BMP), and the macrostructure of the highly porous DBM itself to serve both as an osteoconductive matrix and to signal the patient's tissue and cells to initiate the growth of new bone (osteoinduction). The formulation is used primarily in contact with bleeding bone. This condition is created either from trauma or a surgical procedure, that may involve drilling, sawing, grinding or scraping the bone to achieve a bleeding condition. In surgery, the bone is traumatized or surgically cut exposing blood capillaries, Haversian canals (micro-channels in the bone), periosteum (the protective tissue lining around bone), muscle and other structures in the surgical site. Bleeding at the site is considered a favorable condition to enhance healing of the wound site by bringing to the site the patient's own cytokines, i.e., proteins and other molecules which are the body's mechanism to carry out the healing process. Any interference with the blood cell mechanism would be considered non-biocompatible and an adverse outcome.

In order for the DBM to be osteoinductive, interference either from the traumatized cells or the formulation must be at a minimum, i.e., a biocompatible condition should be established and maintained. Several specific properties have been established in the composition formulation to create a functional material. These properties pertain to both physical characteristics and to the achieving of a biocompatible or physiologically friendly condition.

It an object of the invention to provide a flexible strip which can be used in spinal fusion.

It is an object of the invention to utilize a mineralized, partially demineralized or fully demineralized preformed bone structure of a shape that is useful to facilitate insertion into a limited area.

It is also an object of the invention to create a preformed bone defect material which can be easily handled by the physician and does not degenerate when contacting blood flow at the surgical site.

It is another object of the invention to create a bone defect material which does not interfere with healing at the wound site and promotes faster bone formation.

It is still another object of the invention to provide a preshaped bone defect form which can be used at the point of surgery.

These and other objects, advantages, and novel features of the present invention will become apparent when considered with the teachings contained in the detailed disclosure along with the accompanying drawings constitute part of this specification and illustrate the embodiment of the invention which together with the description serve to explain the principles of the invention.

### BRIEF DESCRIPTION OF THE DRAWINGS

FIG. 1 is a perspective view of a composition strip of the present invention.

### DESCRIPTION OF THE INVENTION

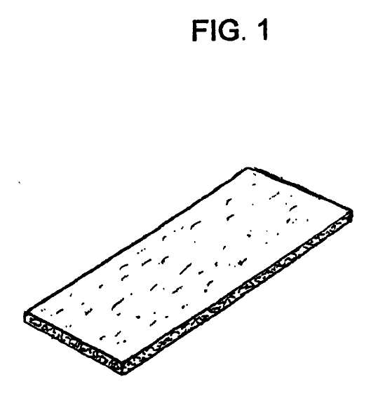

The present invention and best mode as shown in FIG. 1 is directed towards a shaped implant of partially demineralized bone material (DBM) formulation having a residual calcium content ranging between about 3 to about 10%, preferably 4 to 6% mixed with a gelatin, hydrogel and a phosphate buffer.

The use of the term shaped as applied to the osteoimplant, means a predetermined or regular form or configuration in contrast to an indeterminate or vague form or configuration and by way of example would be characteristic to a wedge, cylinder, disk, plate sheet, tube and the like.

The term demineralization as used in relation to treatment of bone up through at least the middle of the 1990's was construed by those skilled in the art to mean that all or substantially all of the mineral content of bone was removed leaving the bone with a residual calcium approaching 0.0% but less than 0.01%. In the late 1990's the term demineralized was used to describe bone which had been subjected to demineralization and had a greater residual calcium content. The terms “fully demineralized” as applied to the bone particles refers to bone particles possessing less than 2%, preferably less than about 1% by weight percent of their original inorganic mineral content; “partially demineralized” is used to refer to bone after mineral removal, which has residual calcium left therein in an amount of at least 3% by weight but less than 10% and “minimally demineralized” is used to refer to bone particles possessing at least about 90% by weight of their original inorganic mineral content. The unmodified term “demineralized” as applied to the bone particles is intended to cover any one or combinations of the foregoing described types of demineralized bone particles.

The DBM is prepared by soaking the bone segments for several minutes in a container with enough sterile ethanol to cover the tissue. The bone segments are milled and placed in a sieve to size the milled bone to 100-800 microns or coarse ground to achieve cortical/cancellous chips in the form of irregularly shaped polyhedra with an edge dimension up to 5 mm. The milled bone material is placed in mixing container and cleaned with a 5:1 ratio of 3% Hydrogen Peroxide and stirred for 15 minutes, removed and rinsed with a minimum of 3000 ml of sterile water. The rinsed bone powder is placed back into the cleaned mixing container and at least 1000 ml of 70% sterile ethanol is added and the solution is mixed for 30 minutes. The bone powder is then transferred into a No. 70 sieve and an open vacuum is applied to the bottom of the sieve and the bone powder is dried for 20 minutes. The dried bone powder is transferred to the demineralization process where it is weighed. The bone weight in grams is compared to a chart which determines the acid volume to be applied which is approximately 1 gram equals approximately 16 ml of acid. The bone powder is mixed with 0.6N HCl for about 2½ hours to achieve maximum bone powder surface engagement with the HCl to remove most of the mineral content. The bone powder can be left for a longer period of time to fully demineralize the bone powder.

When cortical/cancellous bone chips are used the bone chips are transferred to the demineralization process where the same is weighed. Bone chips are mixed with 0.6N HCl at a 1:16 ratio and treated for a longer time of up to 8 hours. Alternatively cortical/cancellous bone chips are mixed with 0.6N HCl which is calculated at a 1:30 ratio and treated for 3 to 5 hours to control the residual calcium content in the range of 4% to 8%. Similarity the bone chips can be left in acid for a longer period to time to achieve fully demineralized bone product.

The bone material is then rinsed with water and 800 ml of sodium phosphate dibasic buffer solution is added to the mixture and the mixture is stirred for about 1 hour to stabilized the pH at around 7.0. The buffered bone powder is then rinsed with sterile water several times leaving a preferred residual calcium content ranging from about 3.0% to about 8% by dry weight of the bone with an optimum preferred residual calcium content of 4% to 6%.

The combination of the respective sized components of demineralized, lyophilized, allograft bone when mixed with a carrier of PSB and gelatin produces a osteoinductive bone defect material which can be molded into any desired shape to form a solid construct. This construct is not readily dissolved and washed away by the blood and fluids at the wound site and thus will present osteoinductivity.

The amount of DBM is maximized to achieve the optimum balance of osteoinductivity and physical handling properties. Too much matrix bone creates a gritty or sandy condition in which the DBM is not ideally enclosed by the surrounding viscous matrix and the DBM bone particles would be too easily washed away. Conversely, if the bone concentration is too low, the osteoinductivity would be less than optimum. Bone concentration in the implant can be in the range of about 30% to about 50% prior to crosslinking and from about 35% to about 65% after crosslinking and gelatin is present in the range of about 5% to about 20% prior to crosslinking and from about 7% to about 25% after crosslinking upon completion of the lyophilization process. Lyophilization is conducted under conditions known in the art, namely an initial shelf temperature of from about −20° to about −55° C., preferably −40° C. for 4 hours, with the temperature raised to +35° C. for 28 hours, with the last 29 hours being under a vacuum of about 350 mTorr. The composition then sits at ambient temperature for 1 hour. The present invention can additionally use HA having a molecular weight of about 7.0×10⁵−3.0×10⁶ Daltons. The present formulation uses a 700,000 Dalton molecular weight hydrogel (sodium hyaluronate or HA). The terms HA or sodium hyaluronate should be construed throughout this application as encompassing sodium hyaluronate, hyaluronic acid, pharmaceutically acceptable salts of hyaluronic acid, derivatives of hyaluronic acid and pharmaceutically acceptable salts of hyaluronic acid derivatives and mixtures thereof. This HA material is used at a 10-25% concentration in the gelatin and 20% to 35% phosphate buffered saline.

The gelatin powder is mixed with sodium phosphate dibasic buffer (pH=9) on a warm plate until the mixture is uniform and completely dissolved. While the gelatin is mixing with the buffer, DBM and the Hyaluronan carrier are mixed separately until uniformly mixed.

The DBM/Hyaluronan carrier mixture is combined with the gelatin-buffer solution. The formulation is equilibrated a warm temperature and stirred to ensure uniformity. The formulation is equilibrated at warm temperature and stirred to ensure uniformity. The formulation is compressed on a warmer roller and remixed, then compressed for a second time. The compressed sheet of DBM—carrier mixture is cut into strips of various sizes and lyophilized for 36 hours plus or minus 8 hours. After lyophilization, the strips are re-hydrated with USP purified water to its original weight.

Lesser molecular weight hydrogels can also be used. Such lesser weight hydrogels are 1) Chitosan about 10,000 to 300,000 Daltons; 2) Sodium Alginate about 10,000 to 300,000 Daltons; 3) Dextran about 40,000 Daltons; 4) carboxymethylcellulose (CMC) about 20,000 to 40,000 Daltons and 5) hydroxypropylmethylcellulose (HPMC) about 20,000 to 40,000 Daltons. Another non hydrogel substances which can be used is Collagen.

The natural condition for blood plasma as well as synovial fluid, cerebrospinal fluid, aqueous humor (fluid within the globe of the eye) is at a pH of 7.3-7.4 (reference, Principles of Biochemistry, Chapters 34 & 35; White, Handler and Smith, McGraw Hill, N.Y., 1964). At very slight changes in pH, blood cells will shift their equilibrium of hemoglobin. This hemoglobin concentration will change over the small pH range of 7.3 to 7.7 (White et al p. 664). In addition, at significantly lower pH values in the acidic range, protein molecules will denature, i.e., degrade. Thus, it is important to maintain any surgical implant which is intimate contact with blood at a biocompatible condition of about pH 7.2-7.4.

It is important to note that the body has many complex and redundant mechanisms to maintain its biochemical balance. The blood pH can be adjusted by several means to its normal, physiologic pH. Hence the presence of a non-physiologic material at the site of a bleeding bone wound will eventually be overcome and any non-biocompatible condition will return to normal pH. It is a teaching of this invention that the preferred formulation will start out and maintain physiologic pH without stressing the body's biochemical mechanisms when the bone composition material is applied at the wound site.

In achieving physiologic pH, the formulation uses a phosphate buffer based on an aqueous system of the two phosphate anions, HPO₄⁻² and H₂PO₄⁻¹. This buffer system is used to neutralize the acid used to demineralize the bone. It is important to neutralize the acid (hydrochloric acid) used to demineralize the bone so as to assure that there is no residue of this very strong acid which could overwhelm the buffering capacity of the phosphate system.

The pH is adjusted to the physiologic 7.2-7.4 pH by using either or both of dibasic sodium phosphate or monobasic sodium phosphate and adjusting the solution with saline, i.e., a sodium chloride solution. The sodium chloride is chosen instead of only water so as to control the final osmolality of the formulation to preclude dehydration of the surrounding cells.

The present invention uses sodium salts of the phosphate buffer. This is to create an equilibrium system at the wound site which will draw in calcium ions necessary to grow new bone. The mechanism to achieve this is based on the LeChatelier corollary to the Principle of Chemical Equilibrium When a factor (temperature, pressure, concentration, etc.) determining the equilibrium of a system is altered the system tends to change in such a way as to oppose and partially annul the alteration in this factor. (reference, General Chemistry, McCutcheon, Seltz and Warner, Van Nostrand N.Y., 1944; p. 248).

The buffer solution will assist in stimulating the formation of bone growth at a bone defect site at a faster rate than a composition without such a buffer. Studies have shown that the presence of phosphate ions accelerates the formation of hydroxyapatite, the principle component of bone. Fulmer, M. T. et al “Effects of Na2HPO4 and Na H2PO4 on hydroxyapatite formation,” J. Biomed Maters, Res., Vol. 27 1095-1102 (1993)

This principal manifests at the bone wound site as follows: The buffer introduced contain % sodium and phosphate ions which will remain in solution due to the high solubility of sodium phosphate. Calcium ions in the extracellular fluid will react with the phosphate ions to result in the precipitation of insoluble calcium phosphate salt. More phosphate ions will ionize from the associated state of the phosphate buffer to introduce more phosphate ions that will, in turn react with more calcium and precipitate yet more insoluble calcium phosphate. The calcium phosphate will deposit at the wound site where the buffered formulation was placed by the surgeon. This results in an increase in the presence of calcium at the wound site. The bone regeneration mechanism will utilize calcium starting 7-10 days after the wound starts healing by the well-known osteochondral healing mechanism. Hence, the selection of the sodium phosphate buffer to achieve the physiologic pH provides a means to increase the calcium concentration in the precise location where calcium will be needed to grow new bone.

Thus, the invention induces the presence of soluble calcium at the bone defect site. This will encourage new bone growth through the normal biochemical mechanism. Soluble calcium can be attracted to the surgical site by using a sodium phosphate buffer of pH 6.8-7.2 in lieu of isotonic saline. The phosphate buffer attracts calcium cations to the site from the surrounding healthy bone and creates an equilibrium concentration of the calcium precisely at the site of healing where it is most desirable to grow new bone.

At low osmolality, the extra cellular environment at the wound site would be in a state of hypotonicity and result in the inflow of large quantities of water to the cells and blood cells at the wound site to normalize the osmotic pressure. This will result in a greater than optimum degree of hydration of the cells and inhibit wound healing in general and bone growth in particular. Hemolysis may occur due to excess fluid in the cells.

Sodium hyaluronate in the form of the sodium salt is generally described as a glycosaminoglycan (GAG). It is envisioned that suitable amounts of bone morphogenic proteins (BMP) can be added to the composition at any stage in the mixing process prior to lyophilization to induce accelerated healing at the bone site. BMP directs the differentiation of pluripotential mesenchymal cells into osteoprogenitor cells which form osteoblasts. The ability of freeze dried demineralized cortical bone to transfer this bone induction principle using BMP present in the bone is well known in the art. However, the amount of BMP varies in the bone depending on the age of the bone donor and the bone processing. Sterilization is an additional problem in processing human bone for medical use as boiling, autoclaving and irradiation over 2.0 Mrads is sufficient to destroy or alter the BMP present in the bone matrix.

In conducting experiments, it was found that a preformed bone product was obtained when a composition of demineralized allograft bone in a phosphate buffered saline and gelatin carrier was lyophilized to obtain a shaped structure having cross linked gelatin and 25% to 65% demineralized bone content.

### Examples of the Initial Formulation

In the following examples, the components used to determine the formulation are as follows:
1) Pharmaceutical grade gelatin
2) Phosphate Buffered Saline (PBS) (pH 7.38)—Type I water, monobasic sodium phosphate, dibasic sodium phosphate, sodium chloride
3) DBM
4) HA or sodium hyaluronate as defined above

In the preparation of PBS; 1,000 ml Type I purified water (995 g) was placed on a stir plate. 1.8208 g of monobasic sodium phosphate monohydrate (J. T. Baker lot: 33152) was weighed and transferred into the Type I purified water in a bottle. 14.1541 g dibasic sodium phosphate heptahydrate (Mallinckrudt USP Lot: 7896N18595) was weighed and transferred into the bottle. See Table 1. 2.41904 g sodium chloride (J. T. Baker Lot M21474) was weighed and transferred into the bottle on the stir plate. The solution was mixed until all the salts were dissolved (minimum of 15 minutes).

TABLE 1
Components of PBS

| Component | Actual Weight |
|---|---|
| Monobasic sodium phosphate | 1.821 g |
| Dibasic sodium Phosphate | 14.154 g |
| Sodium Chloride | 2.419 g |

The pH meter (VWR brand model 3000 with Hamilton tiptrode electrode) was calibrated: % slope=96.1. The pH measured was: 7.35. Preparation of Gelatin mixtures (gelatin and PBS): The gelatin mixture for each formulation was prepared at the same time as each formulation. 12 weighing pans were labeled 1-12. 12-250 ml beakers were labeled 1-12. The water bath was turned on and the temperature set at 80° C. The second water bath (QC lab's) was filled partially using Type I water. The temperature was set on this water bath to 40° C. The appropriate amount of gelatin was weighed in each weighing pan. The appropriate weight of PBS was weighed in each beaker. The weights were recorded in Table 2.

TABLE 2
Weights of Components for Gelatin Mixtures

| | Gelatin Mix Required | Gelatin | PBS |
|---|---|---|---|
| Sample | for Formulation | Weight | Weight |
| 1 | 16 g | 4.872 g | 11.130 g |
| 2 | 14 g | 4.261 g | 9.742 g |
| 3 | 12 g | 3.651 g | 8.353 g |
| 4 | 12 g | 3.65 g | 8.351 g |
| 5 | 10 g | 3.042 g | 6.962 g |
| 6 | 10 g | 3.043 g | 6.961 g |
| 7 | 8 g | 2.430 g | 5.571 g |
| 8 | 8 g | 2.432 g | 5.571 g |
| 9 | 6 g | 1.832 g | 4.172 g |
| 10 | 6 g | 1.833 g | 4.174 g |

| | |
|---|---|
| 11 | See table 3 below |
| 12 | See table 3 below |

Note:
Formulation 11 was prepared with sodium hyaluronate and its derivatives (HA) and gelatin mixture composing 40% of the formulation.
Formulation 12 was prepared with Gelatin mixture and glycerol.

TABLE 3
Preparation of Formulations 11 and 12 gelatin mixtures (8 g of each)

| | Formulation 11 | Formulation 12 |
|---|---|---|
| Component | Actual Weight | Actual Weight |
| Gelatin | 2.432 g | 1.824 g |
| PBS | 3.571 g | 5.456 g |
| Glycerol | NA | 0.721 g |
| Paste HA | 2 g | NA |
| Total prepared | 6 g + 2 g | 8 g |

Table 4 is a description of the 12 samples of crosslinked bone prepared.

TABLE 4
Description of Formulations

| Sample # | Gelatin Mixture | DBM | Paste HA |
|---|---|---|---|
| 1 | 80% | 20% | 0% |
| 2 | 70% | 20% | 10% |
| 3 | 60% | 40% | 0% |
| 4 | 60% | 30% | 10% |
| 5 | 50% | 50% | 0% |
| 6 | 50% | 40% | 10% |
| 7 | 40% | 60% | 0% |
| 8 | 40% | 40% | 20% |
| 9 | 30% | 70% | 0% |
| 10 | 30% | 60% | 10% |
| 11 | 40% | 60% | — |
| 12 | 40% | 60% | 0% |

Weighing pans were labeled 1-12. (weighing pans were labeled for the gelatin, DBM, and sodium hyaluronate or HA (when needed). A labeled beaker containing the weighed PBS was placed in the 80° C. water bath. The gelatin (in the appropriately labeled weighing pan) was transferred into a beaker in the water bath. The gelatin mixture was mixed with a spatula. The cover was placed on the water bath for approximately 5 minutes. After approximately 5 minutes, the cover was removed and the gelatin mixture was stirred until all the gelatin was dissolved (about 1-2 minutes of stirring after the 5 minutes). The beaker containing the gelatin mixture was transferred into the 40° C. water bath. The gelatin was continued to be stirred with a spatula in the 40° C. water bath for 1-2 minutes. The robo-thermometer was used to monitor the temperature of the gelatin. When the temperature of the gelatin reached about 40° C. (and remained constant), the DBM (and hydrogel such as HA if required) were added to the gelatin. The weights were recorded in table 5.

TABLE 5
Actual Weights of components

| Sample | | | | Grams of | Grams of | Grams | Total |
|---|---|---|---|---|---|---|---|
| # | Gelatin | DBM | HA | Gelatin Mix | DBM | of HA | Prepared |
| 1 | 80% | 20% | 0% | 16.00 | 4.00 | 0 | 20 g |
| 2 | 70% | 20% | 10% | 14.00 | 4.00 | 2.00 | 20 g |
| 3 | 60% | 40% | 0% | 12.00 | 8.00 | 0 | 20 g |
| 4 | 60% | 30% | 10% | 12.00 | 6.00 | 2.00 | 20 g |
| 5 | 50% | 50% | 0% | 10.00 | 10.00 | 0 | 20 g |
| 6 | 50% | 40% | 10% | 10.00 | 8.00 | 2.00 | 20 g |
| 7 | 40% | 60% | 0% | 8.00 | 12.00 | 0 | 20 g |
| 8 | 40% | 40% | 20% | 8.00 | 8.00 | 4.00 | 20 g |
| 9 | 30% | 70% | 0% | 6.00 | 14.00 | 0 | 20 g |
| 10 | 30% | 60% | 10% | 6.00 | 12.00 | 2.00 | 20 g |
| 11* | 40% | 60% | 0% | 6.00 | 12.00 | 2.00 | 20 g |
| 12* | 40% | 60% | 0% | 8.00 | 12.00 | 0 | 20 g |

The formulation was mixed with a spatula until there wasn't any dry bone. The formulation was scooped from the beaker with a spatula and spread (evenly) over a microscope slide. Another slide was placed on top of the formulation. The two slides were evenly pressed together to form the desired thickness of the bone gel sample. The sample was allowed to cool (around room temperature). The edges sticking out of the slides were cut off using a scalpel. The top glass slide was carefully removed from the formulation. The formulation was removed from the bottom slide (it peeled right off the slide). Each formulation was placed into a zip lock bag labeled Gelatin formulation and sample #. Some formulations were too sticky to be placed on the glass slides. These formulations were “rolled out” with a 4-liter amber glass bottle. The rolled pieces were also cut with a scalpel into sheets. They were also placed in plastic bags labeled formulation number. The formulations with the higher DBM concentrations of 60% and over appeared to be dry. Formulation 9 was so dry that all the DBM did not even mix with the gelatin mixture. The formulations with HA appeared mold better to a slide than did the samples without HA. Table 6 shows the percentages of each formulation.

TABLE 6
Percentages of each component per formulation

| | % | % | % | % | % | Total |
|---|---|---|---|---|---|---|
| Sample | Gelatin | PBS | DBM | HA | Glycerol | Prepared |
| 1 | 24.4% | 55.7% | 20% | 0% | 0% | 20 g |
| 2 | 21.3% | 48.7% | 20% | 10% | 0% | 20 g |
| 3 | 18.3% | 41.8% | 40% | 0% | 0% | 20 g |
| 4 | 18.3% | 41.8% | 30% | 10% | 0% | 20 g |
| 5 | 15.2% | 34.8% | 50% | 0% | 0% | 20 g |
| 6 | 15.2% | 34.8% | 40% | 10% | 0% | 20 g |
| 7 | 12.2% | 27.9% | 60% | 0% | 0% | 20 g |
| 8 | 12.2% | 27.9% | 40% | 20% | 0% | 20 g |
| 9 | 9.2% | 20.9% | 70% | 0% | 0% | 20 g |
| 10 | 9.2% | 20.9% | 60% | 10% | 0% | 20 g |
| 11 | 12.2% | 17.9% | 60% | 10% | 0% | 20 g |
| 12 | 9.1% | 27.3% | 60% | 0% | 3.6% | 20 g |

### EXAMPLES

In each of the Examples 1 through 12, the samples (approximately 1″×1″×⅛″) were lyophilized for 33 hours. After the freeze drying period, between 0.1 and 8% water were left in the lyophilized samples. While the DBM particle size was 250-812 micron, a size substitution of 100 to 850 microns would not change the composition.

### Example 1

A cross linked gelatin bone composition of 80% Gelatin mixture and 20% DBM.

4.87 g of gelatin (Pharmaceutical grade gelatin) was mixed with 11.30 g PBS (phosphate buffered saline pH=7.35) in an 80° C. water bath until the mixture was uniform (gelatin was completely dissolved) for a total of 16 g of gelatin mixture. The gelatin mixture was cooled to 40° C. in a separate water bath. 4 g of DBM (demineralized bone matrix power—particle size 250-812 microns) was mixed (with a spatula) into the gelatin mixture (at 40° C.). The formulation was flattened, cooled to room temperature, and cut into sheets using a scalpel. A total of 20 g of gelatin bone was prepared consisting of 20% DBM in 80% gelatin mixture. The formulation was wet with PBS and evaluated before freeze-dried. This formulation was flexible, highly elastic, and had strong tare. After freeze drying, the tissue was re-hydrated with 10 ml PBS and by 40 minutes, the tissue form was completely flexible.

### Example 2

A cross linked gelatin bone formulation of 70% gelatin mixture, 20% DBM, and 10% paste HA. 4.26 g of gelatin (Pharmaceutical grade gelatin) was mixed with 9.74 g PBS (phosphate buffered saline pH=7.35) in an 80° C. water bath until the mixture was uniform (gelatin was completely dissolved) for a total of 14 g of gelatin mixture. The gelatin mixture was cooled to 40° C. in a separate water bath. 2 g of paste HA (Sodium Hyaluronate—paste carrier) was stirred into the gelatin mixture (at 40° C.). 4 g of DBM (demineralized bone matrix power—particle size 250-812 microns) was mixed (with a spatula) into the gelatin mixture with HA (at 40° C.). The formulation was flattened, cooled to room temperature, and cut into sheets using a scalpel. The gelatin bone formulation (20 g) consisted of 20% DBM, 70% gelatin mixture and 10% paste HA. The formulation was wet with PBS and evaluated before freeze-dried. Example 2 was nice and flexible. After freeze drying, the tissue was re-hydrated with 10 ml PBS and at 60 minutes, the tissue form was slightly flexible, intact, and uniform with a little loose bone at corners.

### Example 3

A cross linked gelatin bone formulation of 60% gelatin mixture and 40% DBM.

3.65 g of gelatin (Pharmaceutical grade gelatin) was mixed with 8.35 g PBS (phosphate buffered saline pH=7.35) in an 80° C. water bath until the mixture was uniform (gelatin was completely dissolved) for a total of 12 g of gelatin mixture. The gelatin mixture was cooled to 40° C. in a separate water bath. 8 g of DBM (demineralized bone matrix power—particle size 250-812 microns) was mixed (with a spatula) into the gelatin mixture (at 40° C.). The formulation was flattened, cooled to room temperature, and cut into sheets using a scalpel. A total of 20 g of gelatin bone was prepared consisting of 40% DBM in 60% gelatin mixture. The formulation was wet with PBS and evaluated before freeze-dried. Formulation 3 was very flexible, much thicker than examples 1 and 2, holds together nicely, and is stiffer and much less flexible than examples 1 and 2. After freeze drying, the tissue was re-hydrated with 10 ml PBS and at 60 minutes, it was very stiff and had loose bone around the corners.

### Example 4

A cross linked gelatin bone formulation of 60% gelatin mixture, 30% DBM, and 10% paste HA.

3.65 g of gelatin (Pharmaceutical grade gelatin) was mixed with 8.35 g PBS (phosphate buffered saline pH=7.35) in an 80° C. water bath until the mixture was uniform (gelatin was completely dissolved) for a total of 12 g of gelatin mixture. The gelatin mixture was cooled to 40° C. in a separate water bath. 2 g of paste HA (Sodium Hyaluronate—paste carrier) was stirred into the gelatin mixture (at 40° C.). 6 g of DBM (demineralized bone matrix power—particle size 250-812 microns) was mixed (with a spatula) into the gelatin mixture with HA (at 40° C.). The formulation was flattened, cooled to room temperature, and cut into sheets using a scalpel. The gelatin bone formulation (20 g) consisted of 30% DBM, 60% gelatin mixture and 10% paste HA. The formulation was wet with PBS and evaluated before freeze-dried. Example 4 was much more flexible than Example 3 and it was pretty strong and elastic. After freeze drying, the tissue was re-hydrated with 10 ml PBS and at 60 minutes, it was flexible, intact, and uniform.

### Example 5

A cross linked gelatin bone formulation of 50% gelatin mixture and 50% DBM.

3.04 g of gelatin (Pharmaceutical grade gelatin) was mixed with 6.96 g PBS (phosphate buffered saline pH=7.35) in an 80° C. water bath until the mixture was uniform (gelatin was completely dissolved) for a total of 10 g of gelatin mixture. The gelatin mixture was cooled to 40° C. in a separate water bath. 10 g of DBM (demineralized bone matrix power—particle size 250-812 microns) was mixed (with a spatula) into the gelatin mixture (at 40° C.). The formulation was flattened, cooled to room temperature, and cut into sheets using a scalpel. A total of 20 g of gelatin bone was prepared consisting of 50% DBM in 50% gelatin mixture. The formulation was wet with PBS and evaluated before freeze-dried. Example 5 was strong, but brittle and not flexible. The example cracked. After freeze drying, the tissue was re-hydrated with 10 ml PBS and at 60 minutes, the core piece was very stiff and it was breaking apart.

### Example 6

A cross linked gelatin bone formulation of 50% gelatin mixture, 40% DBM, and 10% paste HA 3.04 g of gelatin (Pharmaceutical grade gelatin) was mixed with 6.96 g PBS (phosphate buffered saline pH=7.35) in an 80° C. water bath until the mixture was uniform (gelatin was completely dissolved) for a total of 10 g of gelatin mixture. The gelatin mixture was cooled to 40° C. in a separate water bath. 2 g of paste HA (Sodium Hyaluronate—paste carrier) was stirred into the gelatin mixture (at 40° C.). 8 g of DBM (demineralized bone matrix power—particle size 250-812 microns) was mixed (with a spatula) into the gelatin mixture with HA (at 40° C.). The formulation was flattened, cooled to room temperature, and cut into sheets using a scalpel. The gelatin bone formulation (20 g) consisted of 40% DBM, 50% gelatin mixture and 10% paste HA. The formulation was wet with PBS and evaluated before freeze-dried. Example 6 was flexible, pretty strong, and slightly brittle. After freeze drying, the tissue was re-hydrated with 10 ml PBS and at 60 minutes, it was slightly flexible with bone loosened around the ends.

### Example 7

A cross linked gelatin bone formulation of 40% gelatin mixture and 60% DBM.

2.43 g of gelatin (Pharmaceutical grade gelatin) was mixed with 5.57 g PBS (phosphate buffered saline pH=7.35) in an 80° C. water bath until the mixture was uniform (gelatin was completely dissolved) for a total of 8 g of gelatin mixture. The gelatin mixture was cooled to 40° C. in a separate water bath. 12 g of DBM (demineralized bone matrix power—particle size 250-812 microns) was mixed (with a spatula) into the gelatin mixture (at 40° C.). The formulation was flattened, cooled to room temperature, and cut into sheets using a scalpel. A total of 20 g of gelatin bone was prepared consisting of 60% DBM in 40% gelatin mixture. The formulation was wet with PBS and evaluated before freeze-dried. Example 7 was highly brittle. It was unacceptable. After freeze drying, the tissue was re-hydrated with 10 ml PBS and at 60 minutes, it was completely broken apart and started breaking apart at 15 minutes.

### Example 8

A cross linked gelatin bone formulation of 40% gelatin mixture, 40% DBM, and 20% HA.

2.43 g of gelatin (Pharmaceutical grade gelatin) was mixed with 5.57 g PBS (phosphate buffered saline pH=7.35) in an 80° C. water bath until the mixture was uniform (gelatin was completely dissolved) for a total of 8 g of gelatin mixture. The gelatin mixture was cooled to 40° C. in a separate water bath. 4 g of paste HA (Sodium Hyaluronate—paste carrier) was stirred into the gelatin mixture (at 40° C.). 8 g of DBM (demineralized bone matrix power—particle size 250-812 microns) was mixed (with a spatula) into the gelatin mixture with HA (at 40° C.). The formulation was flattened, cooled to room temperature, and cut into sheets using a scalpel. The gelatin bone formulation (20 g) consisted of 40% DBM, 40% gelatin mixture and 20% paste HA. The formulation was wet with PBS and evaluated before freeze-dried. Example 8 was flexible and weak. After freeze drying, the tissue was re-hydrated with 10 ml PBS and at 60 minutes, it was disintegrating with a lot of bone coming off of the piece.

### Example 9

A cross linked gelatin bone formulation of 30% gelatin mixture and 70% DBM.

1.83 g of gelatin (Pharmaceutical grade gelatin) was mixed with 4.17 g PBS (phosphate buffered saline pH=7.35) in an 80° C. water bath until the mixture was uniform (gelatin was completely dissolved) for a total of 6 g of gelatin mixture. The gelatin mixture was cooled to 40° C. in a separate water bath. 14 g of DBM (demineralized bone matrix power—particle size 250-812 microns) was mixed (with a spatula) into the gelatin mixture (at 40° C.). The formulation was flattened, cooled to room temperature, and cut into sheets using a scalpel. A total of 20 g of gelatin bone was prepared consisting of 70% DBM in 30% gelatin mixture. Example 9 was too dry to form into a sheet. It couldn't be formed and it returned to the powder form.

### Example 10

A cross linked gelatin bone formulation of 30% Gelatin mixture, 60% DBM and 10% HA.

1.83 g of gelatin (Pharmaceutical grade gelatin) was mixed with 4.17 g PBS (phosphate buffered saline pH=7.35) in an 80° C. water bath until the mixture was uniform (gelatin was completely dissolved) for a total of 6 g of gelatin mixture. The gelatin mixture was cooled to 40° C. in a separate water bath. 2 g of paste HA (Sodium Hyaluronate—paste carrier) was stirred into the gelatin mixture (at 40° C.). 12 g of DBM (demineralized bone matrix power—particle size 250-812 microns) was mixed (with a spatula) into the gelatin mixture (at 40° C.). The formulation was flattened, cooled to room temperature, and cut into sheets using a scalpel. A total of 20 g of gel bone was prepared consisting of 60% DBM in 30% gelatin mixture and 10% HA. The formulation was wet with PBS and evaluated before freeze-dried. This formulation was too brittle. After freeze drying, the tissue was re-hydrated with 10 ml PBS and at 15 minutes, it started to break apart and at 60 minutes, it was almost completely broken apart.

### Example 11

A cross linked gelatin bone formulation of 40% gelatin mixture (15% gelatin mix and 25% HA) and 60% DBM. 2.43 g of gelatin (Pharmaceutical grade gelatin) was mixed with 3.57 g PBS (phosphate buffered saline pH=7.35) in an 80° C. water bath until the mixture was uniform (gelatin was completely dissolved) for a total of 6 g of gelatin mixture. The gelatin mixture was cooled to 40° C. in a separate water bath. 12 g of DBM (demineralized bone matrix power—particle size 250-812 microns) was mixed into the gelatin mixture (at 40° C.). 2 g of paste HA (Sodium Hyaluronate—paste carrier) was stirred into the gelatin mixture. 12 g of DBM (demineralized bone matrix power—particle size 250-812 microns) was mixed (with a spatula) into the gelatin mixture with HA (at 40° C.). The formulation was flattened, cooled to room temperature, and cut into sheets using a scalpel. The gelatin bone formulation (20 g) consisted of 60% DBM, 40% gelatin mixture (15% gelatin mix and 25% HA). The formulation was wet with PBS and evaluated before freeze-dried. Example 11 was very hard, brittle and strong. After freeze drying, the tissue was re-hydrated with 10 ml PBS and at 60 minutes, it was almost completely broken apart with clumps of bones in the PBS.

### Example 12

A cross linked gelatin bone formulation of 40% gelatin mixture and Glycerol, 60% DBM.

1.824 g of gelatin (Pharmaceutical grade gelatin) was mixed with 5.456 g PBS (phosphate buffered saline pH=7.35) and 0.72 g of Glycerol in an 80° C. water bath until the mixture was uniform (gelatin was completely dissolved) for a total of 8 g of gelatin mixture. The gelatin mixture was cooled to 40° C. in a separate water bath. 12 g of DBM (demineralized bone matrix power—particle size 250-812 microns) was mixed into the gelatin mixture (at 40° C.). The formulation was flattened, cooled to room temperature, and cut into sheets using a scalpel. The gelatin bone formulation (20 g) consisted of 60% DBM, 40% gelatin mixture and glycerol. The formulation was wet with PBS and evaluated before freeze-dried. Example 12 was very brittle, weak and not flexible. After freeze drying, the tissue was re-hydrated with 10 ml PBS and at 60 minutes, it was almost completely broken apart with clumps of bone in the PBS.

Temperature differential of gelatin mixture when mixed with DBM resulted in no apparent change in the composition. The following Examples 13 through 15 did not show that the mixing temperature had any effect on product.

### Example 13

A cross linked bone formulation of 50% gelatin mixture and 50% DBM.

3.04 g of gelatin (Pharmaceutical grade gelatin) was mixed with 6.96 g PBS (phosphate buffered saline pH=7.35) in an 80° C. water bath until the mixture was uniform (gelatin was completely dissolved) for a total of 10 g of gelatin mixture. The gelatin mixture was cooled to 70° C. in a separate water bath. 10 g of DBM (demineralized bone matrix power—particle size 250-812 microns) was mixed (with a spatula) into the gelatin mixture (at 70° C.). The formulation was flattened, cooled to room temperature, and cut into sheets using a scalpel. A total of 20 g of gel bone was prepared consisting of 50% DBM in 50% gelatin mixture.

### Example 14

A cross linked gelatin formulation of 50% gelatin mixture and 50% DBM.

3.04 g of gelatin (Pharmaceutical grade gelatin) was mixed with 6.96 g PBS (phosphate buffered saline pH=7.35) in an 80° C. water bath until the mixture was uniform (gelatin was completely dissolved) for a total of 10 g of gelatin mixture. The gelatin mixture was cooled to 60° C. in a separate water bath. 10 g of DBM (demineralized bone matrix power—particle size 250-812 microns) was mixed (with a spatula) into the gelatin mixture (at 60° C.). The formulation was flattened, cooled to room temperature, and cut into sheets using a scalpel. A total of 20 g of gel bone was prepared consisting of 50% DBM in 50% gelatin mixture.

### Example 15

A cross linked gelatin formulation of 50% gelatin mixture and 50% DBM.

3.04 g of gelatin (Pharmaceutical grade gelatin) was mixed with 6.96 g PBS (phosphate buffered saline pH=7.35) in an 80° C. water bath until the mixture was uniform (gelatin was completely dissolved) for a total of 10 g of gelatin mixture. The gelatin mixture was cooled to 50° C. in a separate water bath. 10 g of DBM (demineralized bone matrix power—particle size 250-812 microns) was mixed (with a spatula) into the gelatin mixture (at 50° C.). The formulation was flattened, cooled to room temperature, and cut into sheets using a scalpel. A total of 20 g of gel bone was prepared consisting of 50% DBM in 50% gelatin mixture.

A number of tests were performed to ascertain maximum DBM concentration which could be mixed to form the composition. A ratio of 70:30 (DBM to gelatin carrier) was found to be unacceptable and the mix could not be flattened because it would not hold together.

The following examples were formed with pharmaceutical grade gelatin Batch #: 90611. Glycerol Anhydrous—J. T. Baker lot: K02640. DBM lots: 490020, 890020.

### Example 16

A cross linked gelatin bone formulation of 60% gelatin mixture and 40% DBM.

5.5 g of gelatin (Pharmaceutical grade gelatin) was mixed with 12.5 g PBS (phosphate buffered saline pH=7.35) in an 80° C. water bath until the mixture was uniform (gelatin was completely dissolved) for a total of 18 g of gelatin mixture. 12 g of DBM (demineralized bone matrix power—particle size 250-812 microns) was mixed into the gelatin mixture (at 80° C.). The formulation was flattened, cooled to room temperature, and cut into sheets using a scalpel. The gelatin bone formulation (30 g) consisted of 40% DBM and 60% gelatin mixture. The formulation was wet with PBS and evaluated before freeze-dried. Example 16 was very flexible and strong. After freeze drying, the tissue was re-hydrated with 10 ml PBS and it was very stiff at 60 minutes, flexible and intact at 4 hours.

### Example 17

A cross linked gelatin bone formulation of 50% gelatin mixture and 50% DBM.

4.6 g of gelatin (Pharmaceutical grade gelatin) was mixed with 10.4 g PBS (phosphate buffered saline pH=7.35) in an 80° C. water bath until the mixture was uniform (gelatin was completely dissolved) for a total of 15 g of gelatin mixture. 15 g of DBM (demineralized bone matrix power—particle size 250-812 microns) was mixed into the gelatin mixture (at 80° C.). The formulation was flattened, cooled to room temperature, and cut into sheets using a scalpel. The gelatin bone formulation (30 g) consisted of 50% DBM and 50% gelatin mixture. The formulation was wet with PBS and evaluated before freeze-dried. Example 17 was less flexible than Example 16, but was still strong enough. After freeze drying, the tissue was re-hydrated with 10 ml PBS and at 60 minutes, there was a little loose bone but it was very stiff, at 4 hours, it was less uniform and somewhat flexible.

### Example 18

A cross linked gelatin bone formulation of 60% gelatin mixture (with glycerol) and 40% DBM. 3.41 g of gelatin (Pharmaceutical grade gelatin) was mixed with 10.23 g PBS (phosphate buffered saline pH=7.35) and 1.36 g of glycerol in an 80° C. water bath until the mixture was uniform (gelatin was completely dissolved) for a total of 15 g of gelatin mixture. 10 g of DBM (demineralized bone matrix power—particle size 250-812 microns) was mixed into the gelatin mixture (at 80° C.). The formulation was flattened, cooled to room temperature, and cut into sheets using a scalpel. The gelatin bone formulation (25 g) consisted of 40% DBM and 60% gelatin mixture. The formulation was wet with PBS and evaluated before freeze-dried. Example 18 was stiffer than Examples 16 and 17 and less elastic, but still flexible and strong enough. After freeze drying, the tissue was re-hydrated with 10 ml PBS and at 60 minutes, there was a little loose bone, very stiff at 4 hours, slightly soft cracks when bent, and disintegrated.

### Example 19

A cross linked gelatin formulation of 50% gelatin mixture (with glycerol) and 50% DBM.

3.41 g of gelatin (Pharmaceutical grade gelatin) was mixed with 10.23 g PBS (phosphate buffered saline pH=7.35) and 1.36 g of glycerol in an 80° C. water bath until the mixture was uniform (gelatin was completely dissolved) for a total of 15 g of gelatin mixture. 10 g of DBM (demineralized bone matrix power—particle size 250-812 microns) was mixed into the gelatin mixture (at 80° C.). The formulation was flattened, cooled to room temperature, and cut into sheets using a scalpel. The gel bone formulation (25 g) consisted of 40% DBM and 60% gelatin mixture. The formulation was wet with PBS and evaluated before freeze-dried. Example 19 was nice, flexible and strong. After freeze drying, the tissue was re-hydrated with 10 ml PBS and after 60 minutes when the flexibility was tested, it broke apart.

The formulation can be used as an adhesive to attach bone tissue to a substrate of a woven, wire or plastic mesh or porous material such as sheets of hyaluronan, implantable mesh and ceramics. This adhesive can be used to attach bone tissue to an existing 3D scaffold. Scaffolds currently on the medical market include calcium phosphate, collagen and poly-lactic acid. The formulation can also be used to hold load-bearing forms in position for short periods of time after implantation. When formed as sheets, the sheets can be used as a gasket between the irregular bone tissue surface and the smooth surface of a fixture and the sheets can be heated and softened to allow malleability at the surgical site. The formulation can be additionally used to fill flexible and nonflexible 3D shapes to create a predetermined shape as for example; pouches, capsules or bags.

The flexible strip 10 shown in FIG. 1 was tested as per the formulations shown in Table 6 using as the gelatin 260 Bloom Type A Low Endo Toxin gelatin.

TABLE 7
Gelbone formulation containing 40% DBM

| Components | Calculated wt. | Actual wt. |
|---|---|---|
| Gelatin | 5.5 g (18.33%) | 5.503 g |
| PBS (pH 7.38) | 12.5 g (41.66%) | 12.504 g |
| DBM | 12 g (40%) | 12.006 g |

TABLE 8
Gelbone formulation containing 50% DBM

| Components | Calculated wt. | Actual wt. |
|---|---|---|
| Gelatin | 4.6 g | 4.602 g |
| PBS (pH 7.38) | 10.4 g | 10.404 g |
| DBM | 15 g | 15.008 g |

### Results:

1. The first gel-bone strip was made containing 40% DBM. The piece was very flexible and also strong.
2. The second gel-bone strip was made containing 50% DBM. The piece was also very flexible and strong.

### Evaluations:

The set of evaluations was for pre-lyo pieces from the 12 formulations shown in Table 6. Out of the 12 formulations, three were the best.
1. The first formulation of Sample 2 was a 70% gel mix, 20% DBM and 10% HA formulation. This piece was considered flexible, and acceptable.
2. The second formulation of Sample 4 was a 60% gel mix, 30% DBM and 10% HA formulation. This piece was considered flexible and acceptable, bends easy, pretty strong and better then the 70%/20%/10% sample.
3. The third formulation of Sample 6 was a 50% gel mix, and 40% DBM 10% HA. This piece was considered flexible, slightly brittle and pretty strong.

Observations of re-hydrated samples were taken of the twelve sample formulations of Table 6. The samples above had the best observations given.

### Conclusion:

The sample containing 60% gelmix/30% DBM/10% HA was the most preferred formulation.
1. The first sample made was a 40% DBM and 60% gelatin mix without paste HA.

TABLE 9
Weights for a 40% DBM and 60% gelatin mix without paste
HA formulation

| | Calculated wt. | | |
|---|---|---|---|
| Components | and percentage | Actual wt. | Comments |
| Gelatin | 3.65 g (18.3%) | 3.650 g | Somewhat hard to mold, |
| PBS (pH 7.38) | 8.35 g (41.8%) | 8.353 g | sticky. Had to wait for it to |
| DBM | 8.0 g (40%) | 8.008 g | cool down a bit in order |
| | | | to mold. Strip came out |
| | | | uniform, but 3 mm thick |
| | | | instead of 2 mm |

2. The second sample was a 40% DBM, 50% gelatin mix, and 10% HA.

TABLE 10
Weights for a 40% DBM, 50% gelatin mix, and 10% HA formulation.

| | Calculated wt. | | |
|---|---|---|---|
| Components | and percentage | Actual wt. | Comments |
| Gelatin | 3.04 g (15.2%%) | 3.043 g | Very good piece, very |
| PBS (pH 7.38) | 6.96 g (34.8%) | 6.969 g | uniform. Was easy to |
| DBM | 8.0 g (40%) | 8.004 g | mix and mold. Best of |
| HA paste | 2.0 g (10%) | 2.001 g | the three |

3. The third sample was a 30% DBM, 60% gelatin mix and 10% RA. The samples with HA looked the best as projected in the previous study.

TABLE 11
Weights for 30% DBM, 60% gelatin mix and 10% HA formulation

| | Calculated wt. | | |
|---|---|---|---|
| Components | and percentage | Actual wt. | Comments |
| Gelatin | 3.65 g (18.3%) | 3.650 g | Sample did not come out as |
| PBS (pH 7.38) | 8.35 g (41.8%) | 8.353 g | clean cut as the other two. |
| DBM | 6.0 g (30%) | 6.002 g | Very gooey after taken out |
| HA paste | 2.0 g (10%) | 2.002 g | of the bath, and before |
| | | | molding. |

The 40% DBM, 50% formulation of the second sample shown in Table 10 rehydrated the fastest.

Three samples with different HA % were made to determine the percentage of HA paste to use.
1. 40% DBM with 50% gelatin-mix and 10% HA
2. 40% DBM with a 38% gelatin-mix and 20% HA
3. 40% DBM with 30% gelatin-mix and 30% HA

The 40% DBM with 50% gelatin-mix and 10% HA, was the best. The one with 30% HA was too weak, and the 20% HA was little better but not as good as the one with 10% HA. This experiment determined that 20% HA or above was not good for the gel-bone snip with this current formulation.

### Re-hydration Test on Samples

The sample formulation with 40% gelatin mix, 38% DBM and 20% HA was the best flexible sample.

Note that the gelatin is freezer milled into a fine powder. The fine powder increase the surface area, which allows for faster dissolving and at a lower temperature of 40° C. The lower temperature melting allowed lowering the temperature at which the DBM came into contact with gelatin mix.

Strip Production Steps
1. Low endo toxin gelatin is milled with a freezer mill
2. All milled particles are passed through a #80 sieve (180 microns)
3. Sterilize gelatin powder at 25-38 Kgy of gamma irradiation.
4. Transfer the gelatin powder and the buffer into a 60 ml bottle. Use a spatula to mix the 2 components together.
5. Once the gelatin is dissolved in the bath (approximately 20 mins), add the DBM and mix with gelatin—Add the HA paste from a syringe to the bottle and mix all the components together.
6. Mix the bone and the binding agent until there is no dry bone left.
7. Place the formulation back into the bath and equilibrate for a minimum of 1 hour.
8. Use a 3″ spatula to remove the formulation from the container and place into a 20 mL cut tip syringe. Compress the formulation in the syringe by facing open end of the syringe down on a flat surface and press the syringe down until the formulation is completely compressed.
9. Deliver the formulation into the mold and use the paper side of a sterile chex-all to cut out strip that will fit into the pockets of the mold. These strips will protect the underside of the formulation when removing from the mold.
10. Press the formulation into the mold with a rolling pin. Use the rolling pin to flatten the formulation in a forward and back motion until the piece is compressed.
11. Release the strips from the mold.
12. Lyophilize for a 36 hour cycle.
13. Re-hydrate each strip with its own wet paper wrap pre-wet or with an amount of water calculated for rehydration.
14. Let the strips sit for about one hour.
15. Package the strips individually in a manager foil pouch and then seal in a pouch of Kapak

The strip formulation comprises a preferred range of about 30% to about 50% DBM and about 45% to about 60% gelatin hyaluronan mixture carrier. The gelatin hyaluronan carrier consists of a range of about 7% to about 17% gelatin, a range of about 10% to about 22% hyaluronan and a range of about 22% to about 32% phosphate buffer. The most preferred formulation consists of a range of about 43% to about 47% DBM and a range of about 53% to about 57% gelatin-hyaluronan mixture carrier. The gelatin-hyaluronan carrier consists of about 10% to about 13% gelatin, about 10% to about 18% hyaluronan and about 24% to about 29% phosphate buffer.

The stiff cross linked material can be made flexible by controlled rehydration to produce a flexible, strong, suturable strip which is useful as a spinal fusion device, particularly for posteriolateral spinal fusion. The basic gelatin/cortical-based DBM/water mixture (“gelbone”) can be formed in a variety of useful shapes and then freeze dried to retain the preformed shape. Thus, blocks, wedges, spheres, ovoid, granules, chips and powder shapes can be used to fill a space in a bony defect. The stiffness of the shapes is useful as they will maintain their stiffness during the insertion phase during the surgery. The stiffness of a wedge, e.g., would facilitate the insertion into a limited space as in an interbody spinal fusion. The stiffened implant would deflect the adjacent tissues creating a space for the dbm material to be placed with a minimum of cutting of the soft tissues in the interbody space. This will limit trauma and bleeding induced by the conventional techniques requiring cutting and dissection. The other shapes are useful for filling load supporting cages for use in spinal fusion.

In conducting experiments, it was found that a bone product with optimal molding and handling properties was obtained when a composition of demineralized allograft bone in a phosphate buffered saline and gelatin carrier was lyophilized to obtain a shaped or unshaped structure having cross linked gelatin and 25% to 65% demineralized bone content ((DBM).

The formulation can be compression molded as a casting, lyophilized and then machine finished to final shape. It is also apparent that the formulation can be molded with cavities created for autogenous tissue, allograft tissue or fluids. The implants can be cut into shapes to fill voids in existing allograft forms, for example the canals in spine spacers and non-allograft medical implants where bone in growth is beneficial.

It is also envisioned that the implant can be molded and machined and/or processed with a load bearing component inserted after processing. It is also envisioned that the implant can be molded or machined into a scaffold or structure to support growth factors, pharmaceuticals or glues that can be sprayed, implanted or applied.

Any number of medically useful substances can be used in the invention by adding the substances to the composition at any steps in the mixing process or directly to the final composition. Such substances include collagen and insoluble collagen derivatives, hydroxy apatite and soluble solids and/or liquids dissolved therein. Also included are antiviricides such as those effective against HIV and hepatitis; antimicrobial and/or antibiotics such as erythromycin, bacitracin, neomycin, penicillin, polymyxin B, tetracycline, viomycin, chloromycetin and streptomycin, cefazolin, ampicillin, azactam, tobramycin, clindamycin and gentamycin and silver salts. It is also envisioned that amino acids, peptides, vitamins, co-factors for protein synthesis; hormones; endocrine tissue or tissue fragments; synthesizers; enzymes such as collagenase, peptidases, oxidases; polymer cell scaffolds with parenchymal cells; angiogenic drugs and polymeric carriers containing such drugs; collagen lattices; biocompatible surface active agents, antigenic agents; cytoskeletal agents; cartilage fragments and peptide growth factors, living cells such as chondrocytes, blood cells, bone marrow cells, mesenchymal stem cells, natural extracts, tissue transplants, bioadhesives, bone morphogenic protein (BMP, (BMP 2, 4, 7), transforming growth factor (TGF-beta), platelet derived growth factor (PDGF), osteopontin, fibroblast growth factor (FGF), insulin-like growth factor (IGF-1); growth hormones such as somatotropin; bone digestors; antitumor agents; fibronectin; cellular attractants and attachment agents; immuno-suppressants, permeation enhancers, e.g. fatty acid esters such as laureate, myristate and stearate monoesters of polyethylene glycol, enamine derivatives, alpha-keto aldehydes can be added to the composition.

While the dry form has significant stiffness, the material will rapidly disaggregate as the gelatin component dissolves in body fluids. This allows the DBM component to initiate the osteoinductive and osteoconductive properties inherent in its composition by virtue of the intrinsic bmp's present in DBM. Hence, a stiff, rigid form can be used to introduce DBM into surgical spaces not readily accessible by the currently available pastes and putties based on dbm.

Another embodiment of the “gelbone” material would be to use cancellous bone rather than the cortical bone described above. The cancellous bone with or without demineralization first would be compressed and mixed with the hyaluronan/gelatin/water components. The mixture is then freeze dried thus producing a stiff composition which when wetted would expand 5-25%. This swellable property would facilitate the filling of preformed spaces in bone voids or between bones as in fracture repair or reshaping bone for cosmetic surgery. The version with demineralized DBM would then initiate the osteoinductive and osteoconductive properties inherent in its structure.

The principles, preferred embodiments and modes of operation of the present invention have been described in the foregoing specification. However, the invention should not be construed as limited to the particular embodiments which have been described above. Instead, the embodiments described here should be regarded as illustrative rather than restrictive. Variations and changes may be made by others without departing from the scope of the present invention as defined by the following claims:

What we claim is:

1. A sterile bone repair strip for application to a bone defect site to promote new bone growth at the site comprising a mixture of osteoinductive bone material in a carrier forming a composition, the bone material ranging from about 30% to about 50% of the weight of the composition of the strip and the carrier comprising a gelatin component dissolved in an buffered aqueous solution ranging from about 45% to about 60% of the weight of the composition of the strip and a hydrogel ranging from about 10% to about 20% of the composition, said composition being lyophilized to achieve a cross linking of the gelatin to obtain a structural stability and a pH ranging from about 6.5 to 7.5.

2. A sterile bone repair strip as claimed in claim 1 wherein said gelatin component ranges from about 7% to about 17% by weight of the composition of the strip prior to lyophilization.

3. A sterile bone repair strip as claimed in claim 1 wherein said aqueous solution is a phosphate buffer ranging from about 20% to about 30% of the composition of said strip prior to lyophilization.

4. A sterile bone repair strip as claimed in claim 1 wherein said bone material is partially mineralized with a calcium content between about 4% to about 8%.

5. A sterile bone repair strip as claimed in claim 1 wherein said aqueous solution comprises at least one of a group consisting of saline and phosphate buffered saline.

6. A sterile bone repair strip as claimed in claim 1 wherein said demineralized bone material ranges from about 40% to about 50% by weight of the composition of said strip prior to lyophilization.

7. A sterile bone repair strip as claimed in claim 1 wherein said hydrogel is a hyaluronan.

8. A sterile repair strip as claimed in claim 7 wherein said hyaluronan is sodium hyaluronate and its derivatives.

9. A sterile bone repair strip as claimed in claim 1 wherein said gelatin is a pharmaceutical grade milled to a range of about 100 microns to 200 microns in size.

10. A sterile bone repair strip as claimed in claim 1 wherein said bone material contains growth factors such as bone morphogenic protein (BMP), (BMP 2, 7), transforming growth factor (TGF-beta), platelet derived growth factor (PDGF), osteopontin, fibroblast growth factor (FGF) and insulin-like growth factor (IGF-1).

11. A sterile bone repair strip as claimed in claim 1 wherein said gelatin component is 260 Bloom Type A low entoxin gelatin.

12. A sterile bone repair strip as claimed in claim 1 including antimicrobial and/or antibiotics such as erythromycin, bacitracin, neomycin, penicillin, polymyxin B, tetracycline, viomycin, chloromycetin and streptomycin, cefazolin, ampicillin, azactam, tobramycin, clindamycin, gentamycin and vitamins.

13. A sterile bone strip as claimed in claim 1 wherein said strips are rehydrated in water and placed in a sterile container.

14. A sterile bone repair strip for application to a bone defect site to promote new bone growth at the site comprising a mixture of osteoinductive bone material in a carrier, the bone material ranging from about 40% to about 50% of the weight of the composition of the strip and the carrier comprising a gelatin component dissolved in an buffered aqueous solution ranging from 40% to about 60% of the weight of the composition of the strip and a hydrogel ranging from 10% to 20% of the weight of the strip, said strip being lyophilized to achieve a cross linking of the gelatin to obtain a structural stability and a pH ranging from about 6.5 to 7.5 and then rehydrated and placed in a sterile container.

15. A sterile bone repair strip as claimed in claim 14 wherein said bone material is partially demineralized and has a residual calcium content ranging from about 4% to about 8%.

16. A sterile bone repair strip as claimed in claim 14 wherein said aqueous gelatin carrier includes a hydrogel comprising at least one of a group consisting of sodium hyaluronate and its derivatives, chitosan, sodium alginate, dextran, carboxymethylcellulose (CMC) and hydroxypropylmethylcellulose (HPMC).

17. A sterile bone repair strip for application to a bone defect site to promote new bone growth at the site comprising a mixture of osteoinductive bone material in a carrier, the bone material ranging from about 40% to about 50% of the weight of the composition and the carrier comprising a gelatin component dissolved in an buffered aqueous solution ranging from 40% to about 60% of the weight of the composition of the strip and a hyaluronan ranging from 10% to 20% of the composition of the strip, said strip being lyophilized to achieve a cross linking of the gelatin to obtain a structural stability and rehydrated to become flexible.

18. A sterile preformed bone implant for application to a bone defect site to promote new bone growth at the site comprising a new bone growth inducing partially demineralized lyophilized allograft bone particles with a residual calcium content of about 4 to 8% in an aqueous gelatin carrier which is lyophilized to remove water content leaving a cross linked gelatin bone structure having a structural stability which is maintained after application to said bone repair site with bone material ranging from about 40% to about 50% of the weight of the composition and the cross linked gelatin ranging from about 7% to about 17% by weight of the composition and a hydrogel taken from a group consisting sodium hyaluronate and its derivatives, chitosan, sodium alginate, dextran, carboxymethylcellulose and hydroxypropylmethylcellulose ranging from 10% to 20% by weight of the composition prior to lyophilization.

19. A sterile preformed bone implant as claimed in claim 18 wherein said allograft bone is compressed cancellous bone.

20. A sterile preformed bone implant as claimed in claim 18 wherein said allograft bone particles have a particle size ranging from about 100 microns to about 850 microns.

21. A method of constructing a cross linked osteoinductive bone repair strip construct comprising the steps of
a. mixing osteoinductive bone material in an aqueous gelatin mixture containing a hydrogel to obtain a formulation;
b. shaping the formulation to a predetermined shape;
c. subjecting said formulation to lyophilization to remove at least 90% of the water from said aqueous gelatin mixture cross linking said gelatin and osteoinductive bone material contained therein to form a solid structure; and
d. rehydrating the lyophilized shaped strip to a flexible condition.

22. A method as claimed in claim 21 wherein said lyophilization is at −40° C. for about 30 to about 35 hours.

23. A method as claimed in claim 21 wherein after said rehydrating step d. is the flexible molded shape is placed in a sterile sealed container.

24. A method as claimed in claim 21 wherein about 0.1 to about 10% of the original water is left in the formulization after lyophilization.

25. A method as claimed in claim 21 wherein said sterile strip is placed in sealed container.
